(19) United States
(12) Patent Application Publication
(10) Pub. No.: US 2021/0386576 A1
(43) Pub. Date: Dec. 16, 2021

(54) Method and System for Medical Emergency First Aid in an Accident using Cooling Cervical Spine Collar and Cooling Protective Helmet

(71) Applicant: Philip Chidi Njemanze, (US)

(72) Inventor: Philip Chidi Njemanze, (US)

(21) Appl. No.: 16/897,273

(22) Filed: Jun. 10, 2020

(51) Int. Cl. A61F 5/055; A61F 7/00

(52) U.S. Cl. CPC A61F 5/055; A61F 7/00; A61F 2007/0093; A61F 2007/0056; A61F 2007/0091; A61F 2007/0012

(57) ABSTRACT

The present invention provides a medical emergency first aid during accidents using cervical spine collar to insert a splint for stabilization, resuscitation, initiation of mild to moderate external hypothermia, and vital signs monitoring in victims of traumatic brain (TBI) and cervical spinal injury (CSI). The cervical spine collar of the present invention is housed in the head rest of the vehicle seat and could be deployed automatically using artificial intelligence (AI) or manually by an assistant. One embodiment incorporates a protective helmet with cooling apparatus to induce mild to moderate external hypothermia of the brain to prevent deleterious effects of TBI.

### CROSS-REFERENCE TO RELATED APPLICATION

U.S. Patent Documents

| Document Number | Date | Name | Classification | Cited by |
|---|---|---|---|---|
| 7,318,834 B2 | 01-2008 | Njemanze | 600/438 | Inventor |
| 6,217,552 B1 | 04-2001 | Barbut et al. | 604/113 | Inventor |
| 6,660,026 B2 | 12-2003 | Larnard et al. | 607/104 | Inventor |
| 6,929,656 B1 | 08-2005 | Lennox | 607/105 | Inventor |
| 7,004,961 B2 | 02-2006 | Wong et al. | 607/105 | Inventor |
| 7,144,418 B1 | 12,2006 | Lennox | 607/105 | Inventor |
| 20020198579 A1 | 12/2002 | Khanna | 607/105 | Inventor |
| 20040143312 A1 | 7/2004 | Samson et al. | 607/105 | Inventor |
| 5,486,208, | 01/1996 | Ginsburg | 607/106 | Inventor |
| 6,558,412 | 05/2003 | Dobak III | 607/105 | Inventor |
| 6,581,400 | 06/2003 | Augustine et al., | 62/259.3 | Inventor |
| 6,197,045 | 03/2001 | Carson | 607/104 | Inventor |
| 6,620,187 | 09/2003 | Carson et al. | 607/104 | Inventor |

### OTHER PUBLICATIONS

1. Adams M, Hutton W. The effect of fatigue on the lumbar intervertebral disc’, J Bone Joint Surg Br 1983; 65-B:199-203.
2. Bernard S A, Gray T W. Buist M D, et al. Treatment of comatose survivors of out-of-hospital cardiac arrest with induced hypothermia. N Engl J Med 2002; 346:557-563.
3. Biering-Sorensen F, Bickenbach J E, El Masry W S, et al. ISCoS-WHO collaboration. International Perspectives of Spinal Cord Injury (IPSCI) report’, Spinal Cord 2011; 49: 679-683.
4. Cassidy J, Spitzer W, Skovron M, et al. The Quebec Task Force on Whiplash-Associated Disorders. Spine 1996; 21:897-878.
5. Colbourne F. Sutherland G. Corbett D. Posttraumatic hypothermia: a critical appraisal with implication for clinical treatment. Mol Neurobiol 1997; 14:171-201.
6. Curatolo M, Bogduk N, Ivancic P C, et al. The role of tissue damage in whiplash-associated disorders. Spine 2011; 36:S309-315.
7. Faul M, Xu L, Wald M M. Coronado V C. Traumatic brain injury in the United States: Emergency Department Visits, hospitalization and deaths, 2002-2006. Atlanta (Ga.): Centers for Disease Control and Prevention, National Center for Injury Prevention.
8. Feng H, Ning G, Feng S, et al. Epidemiological profile of 239 traumatic spinal cord injury cases over a period of 12 years in Tianjin, China. J Spinal Cord Med 2011; 34:388-394.
9. Gunn A J. Cerebral hypothermia for prevention of brain injury following perinatal asphyxia’, published in the Curr Opin Pediatr 2000; 12:111-115.
10. Irazurta J E, Olson J, Kiefaber M P, Wong H. Hypothermia decreases excitatory neurotransmitter release in bacterial meningitis in rabbits. Brain Res 1999; 847:143-148.
11. Jubran A. Pulse oximetry. Crit Care. 2015; 19(1):272. Published 2015 Jul. 16. doi: 10. 1186/s13054-015-0984-8
12. Konda S, Al-Tarawneh I S, Reichard A A, et al. Workers' compensation claims for traumatic brain injuries among private employers-Ohio, 2001-2011’. Am J of Ind Med 2019, 63:156-169.
13. Li F, Liu N, Li H, et al. A review of neck injury and protection in vehicle accidents. Transportation Safety and Environment 2019, 1(2):89-105.
14. Ma H, Sinha B, Pandya R S, et al. Therapeutic hypothermia as a neuroprotective strategy in neonatal hypoxic-Ischemic Brain Injury and Traumatic Brain Injury. Curr Mol Med 2012; 12(10):1282-1296.
15. Metz C, Holzschuh M, Bein T, et al. Moderate hypothermia in patients with severe head injury: cerebral and extracerebral effects. J Neurosurg 1996; 85: 533-541.
16. Mez J, Daneshvar D H, Kiernan P T, et al. Clinicopathological Evaluation of Chronic Traumatic Encephalopathy in Players of American Football. J AMA 2017; 318(4):360-370. doi:10.1001/jama.2017.8334
17. Njemanze P C, Beck O J. MR-Gated Intracranial CSF dynamics: evaluation of CSF pulsatile flow. AJNR 1989; 10:77-80.
18. Oichi Y, Hanakita, Takahashi T et al. Morphological patterns of the anterior median fissure in the cervical spinal cord evaluated by computed tomography after myelography, Neurospine 2018; 15 (4):388-393.
19. Quinlan K P, Annest J L, Myers B, et al. Neck strains and sprains among motor vehicle occupants—United States, 2000. Accident Anal Prev 2004, 36:21-27.
20. Romilly D P, Skipper C S. Seat structural design choices and the effect on occupant injury potential in rear end collisions. SAE Transactions, 2005; 114:1512-1520.
21. Rosner M. Pathophysiology and management of increased intracranial pressure. In book by Andrews B T. (ed.) Neurosurgical intensive care, McGraw Hill Inc., New York, pp. 57-112, 1993.
22. Schubert A. Side effects of mild hypothermia. J Neurosurg Anesthes 1995; 7:139-147.
23. Shiozaki T, Sugimoto H, Taneda M, et al. Effect of mild hypothermia on uncontrollable intracranial hypertension after severe head injury. J. Neurosurg 1993; 79(3):363-368.
24. Song J, Shao J, Qi H H, et al. Risk factors for respiratory failure with tetraplegia after acute traumatic cervical spinal cord injury. Eur Rev Med Pharmaco 2015; 19:9-14.
25. Sterner Y, Gerdle B. Acute and chronic whiplash disorders—a review, published in J

Rehabil Med 2004; 36:193-210.
26. Konda S, Al-Tarawneh I S, Reichard A A, et al. Workers' compensation claims for traumatic brain injuries among private employers—Ohio, 2001-2011. Am J Ind Med 2019; 63:156-169.
27. Viano D C. Role of the Seat in Rear Crash Safety. In Warrendale, P A: Society of Automotive Engineers, 2002.
28. Wang C. et al. Monitoring of the central blood pressure waveform via a conformal ultrasonic device. Nat Biomed Eng 2018; 2:687-695.
29. Watanabe Y, Ichikawa H, Kayama O, et al. Influence of seat characteristics on occupant motion in low-speed rear impacts. Accident Anal Prev 2000; 32: 243-250.
30. Zhao Z, Gu T, Zhang Y, Yu L. Effects of Mild Hypothermia on Low Cardiac Output After Cardiac Surgery. Zhongguo Wei Zhong Bing Ji Jiu Yi Xue, 2012; 24(4):219-221.

### STATEMENT REGARDING FEDERALLY SPONSORED RESEARCH OR DEVELOPMENT

Not Applicable

### REFERENCE TO MICROFICHE APPENDIX

Not Applicable

### BACKGROUND OF THE INVENTION

The present invention provides a medical emergency first aid response during accidents using cervical spine collar to provide stabilization, resuscitation and initiation of mild to moderate hypothermia in victims of traumatic brain (TBI) and cervical spine injury (CSI). In one embodiment, it could be used with protective helmet with cooling apparatus to induce mild to moderate hypothermia to prevent deleterious effects of TBI. In another embodiment, the present invention provides a medical emergency first aid safety system in vehicles during accidents using artificial intelligence (AI) to automatically provide initial assessment, initiate stabilization and resuscitation of TBI and CSI sustained by a person in an accident. The device is designed to function automatically with AI system and also in a manual mode by an operator

The invention when controlled by an AI system comprises AIAutoCoolCollar and AIAutoCoolHelmet, which are used in conjunction or separately to prevent severe effects resulting from CSI and TBI. The AI system on detection of a crash automatically uses the pre-determined anthropometric variables and position-sensing of the head and neck including measurements of neck circumference to insert a splint using an automatic cervical spine collar attached to the head rest of the vehicle seat. Simultaneously inflating a system of inner bladders filled with coolant for cooling the neck and back of the head region, while inflating a mini-airbag from the head rest to prevent hyperextension of the head and cervical spine. The ensuing hyperflexion is prevented by the automatic deployment of the front airbag at the steering. It is adapted for use in automobiles, locomotives and avionics, as well as in sports, construction and in the military.

In the United States, between 2002-2006, the CDC estimated that 1.7 million people sustain a TBI annually as disclosed by Faul M, Xu L, Wald M M. Coronado V C., in an article entitled ‘Traumatic brain injury in the United States: Emergency Department Visits, hospitalization and deaths, 2002-2006’, published by Atlanta (Ga.): Centers for Disease Control and Prevention, National Center for Injury Prevention, of them 52,000 died, 275,000 were hospitalized and 1.365 million nearly 80% are treated and released from the emergency department. TBI is a contributing factor to a third (30.5%) of all injury-related death in the United States. About 75% that occur each year are concussions or other forms of mild TBI. The direct medical costs and indirect costs of TBI, such as lost productivity totaled an estimated $60 billion in the USA in 2000. Almost half a million emergency department visits for TBI are made annually by children ages 0-14 years. According to the CDC, in the United States, 5.3 million Americans are living today with disabilities related to TBI. One of the major public health achievements of the 20th century for the United States is reducing motor vehicle crash deaths. However, mortality and morbidity remain more than 32,000 people and 2 million are injured each year from motor vehicle crashes. Car safety systems help to reduce the more serious injuries. In 2013, the US crash death rate was more than twice the average of other high-income countries. In the US, front seat belt use was lower than in most other comparable countries. Seat belts saved over 12,500 lives in the US in 2013.

Present state-of-the art offers helmet protection only for prevention of TBI. However, even with the use of helmets, the impact from high-velocity head injury still causes brain concussions resulting in neurological sequel. It is desirable to have an immediate intervention after the accident before medical emergency help arrives. Researchers have demonstrated the beneficial effects from therapeutic cooling of the brain with the use of mild induced hypothermia (MIH defined as the maintenance of body temperature at 32° C. to 34° C.). MIH exerts significant neuroprotection and attenuates secondary cerebral insults after TBI. In TBI patients, MIH has been used during the acute “early” phase as prophylactic neuroprotection and in the sub-acute “late” phase to control brain edema or swelling. MIH is effective in reducing elevated intracranial pressure (ICP) and is a valid therapy of refractory intracranial hypertension in TBI patients. It is therefore desirable to incorporate the apparatus for hypothermia into the protective helmets used in sports, motorbikes, car-racing, construction, pilots, military and any other areas where protective helmets are worn for improved safety to prevent deleterious effects of TBI. A review of the statistics in the potential areas of application reveals a widespread need.

TBI is a frequent complication of sports injury. According to the National SAFE KIDS Campaign and the American Academy of Pediatrics in the United States, about 30 million children and teens participate in some form of organized sports. There are over 3.5 million injuries each year, with some loss of time for participation by the participants. At least, 30% of all injuries incurred in childhood are sports-related injuries, although they are mostly sprains and strains. The leading cause of death from a sports-related injury is a brain injury. TBI among American children represent about 21% of all injuries. About half of head injuries sustained in sports or recreational activities occur during bicycling, skateboarding, or skating incidents. About 775,000 children, ages 14 and younger, are treated in hospital emergency rooms for sports-related injuries each year. Most of the injuries occurred as a result of falls, being struck by an object, collisions, and overexertion.

In the United States in professional sports up to 3.8 million sports-related concussions occur each year. In a landmark study, on TBI in American Football, it was demonstrated that 110 out of 111 deceased NFL players had chronic traumatic encephalopathy (CTE), a degenerative brain disorder associated with repetitive head trauma. Several other studies have linked CTE to suicidal behavior, brain degenerative diseases and disorders such as dementia, depression and Parkinson's disease. Professional athletes are at higher risk for CTE because of frequent occurrence of concussions and other traumatic brain injuries. In 2016, the NFL officially for the first time acknowledged the link between CTE and American football. Researchers have examined the brains of 202 deceased people donated to Boston University, who had played football at various levels, from high school to the NFL. In 87% of the brains of deceased players, CTE was established in the players according to an article by Mez J, Daneshvar D H, Kiernan P T, et al., entitled ‘Clinicopathological evaluation of chronic traumatic encephalopathy in players of American football’, published in JAMA, 2017, vol. 318(4), pp. 360-370.

According to the Insurance Institute for Highway Safety and National Highway Traffic Safety Administration (NHTSA), 5,286 people died from motorbike accidents in 2015. Most motorbike accident deaths occurred as a result of head injuries. In a study that included 6,646 motorcyclists who had suffered head injuries resulting from accidents, the unhelmeted motorcyclists who sustained head injuries were 8.1% compared to 5.3% in the helmeted motorcyclists. It concluded that wearing helmets was 35% effective at minimizing head injuries when motorbike crashes take place.

In the United States, according to the National Institute for Occupational Safety and Health (NIOSH), during 2003 to 2010, in the construction industry, there were 2,210 deaths because of TBI (a rate of 2.6 per 100,000 FTE workers) according to an article by Konda S, Al-Tarawneh I S, Reichard A A, et al., entitled ‘Workers’ compensation claims for traumatic brain injuries among private employers—Ohio, 2001-2011’, published in American Journal of Industrial Medicine, 2019, vol. 63, pp. 156-169. NIOSH established that, these deaths represented 25% of all construction fatalities and 24% of all occupational TBI fatalities during the same period. Falls, especially from roofs, ladders, and scaffolds, led to >50% of fatal work-related TBIs.

Nevertheless, traffic accidents are the main cause of head and neck injuries that often result in high mortality and long-term morbidity with high socio-economic cost. Annually, in the United States there are more than 800,000 cases of vehicle accidents involving neck injuries with an estimated cost of over $5.2 billion as disclosed by Viano D C., in an article entitled ‘Role of the seat in rear crash safety., published in Warrendale, P A: Society of Automotive Engineers, 2002. The most frequent injury is the whiplash neck injury. Whiplash may involve ligament sprain, disc injury, and muscle strain. In some cases, a herniated disc or vertebral dislocation in the cervical spine may irritate spinal nerve roots or less frequently involve the spinal cord, causing nerve symptoms. Majority of the whiplash occurs during low-velocity rear-end vehicle collisions. In developed countries such as the United States as disclosed by Romilly D P, Skipper C S., in an article entitled ‘Seat structural design choices and the effect on occupant injury potential in rear end collisions’, published in SAE Transactions, 2005; vol. 114, pp. 1512-1520, and Germany, 90% of injuries in rear-end crashes are neck injuries from rear impacts at speeds of less than 25 km/h as disclosed by Watanabe Y, Ichikawa H, Kayama O, et al., in an article entitled ‘Influence of seat characteristics on occupant motion in low-speed rear impacts’, published in Accident Anal Prev 2000; vol. 32, pp. 243-250. There are significant gender differences with more women than men suffering from whiplash (1.5:1) as disclosed by Quinlan K P, Annest J L, Myers B, et al., in an article entitled ‘Neck strains and sprains among motor vehicle occupants—United States, 2000’, published in Accident Anal Prev 2004, vol. 36, pp. 21-27, with greater long-term morbidity as disclosed by Cassidy J, Spitzer W, Skovron M, et al., in an article entitled ‘The Quebec task force on whiplash-associated disorders’, published in Spine 1996, vol. 21, pp. 897-878. In adolescents and adults the annual incidence is between 3-6% as disclosed by Adams M, Hutton W., in an article entitled ‘The effect of fatigue on the lumbar intervertebral disc’, published in J Bone Joint Surg Br 1983, vol. 65-B, pp. 199-203.

In rear-end impact accidents, cervical spine at first suffers a sudden forceful hyperextension followed by hyperflexion resulting in cervical soft tissues sprains or strains. Several symptoms may result which are described as whiplash-associated disorders (WAD), and may include headaches, dizziness, forgetfulness and emotional/psychological disturbances as disclosed by Sterner Y, Gerdle B, in an article entitled ‘Acute and chronic whiplash disorders—a review, published in J Rehabil Med 2004; vol. 36, pp. 193-210. There are several possible sites for this injury: facet joints, spinal ligaments, intervertebral discs, vertebral arteries, dorsal root ganglia and neck muscles as disclosed by Curatolo M, Bogduk N, Ivancic P C, et al., in an article entitled ‘The role of tissue damage in whiplash-associated disorders’, published in Spine 2011; vol. 36, pp. S309-315.

Traffic accidents may cause cervical fracture characterized by vertebral fracture, and cervical dislocation, which may lead to spinal instability. In severe dislocation the cervical bone may fully displace forward and lock in this position in a complication referred to as ‘jumping’. In this case, the ligaments may rupture completely. Cervical dislocations may cause spinal cord injury (SCI) and most patients would require surgery. SCI results from the sudden forceful damage to the spinal nerves, leading to temporary or permanent paralysis, bladder and bowel dysfunction and autonomic imbalance, among other consequences as disclosed by Biering-Sorensen F, Bickenbach J E, El Masry W S, et al., in an article entitled ‘ISCoS-WHO collaboration. International Perspectives of Spinal Cord Injury (IPSCI) report’, published in Spinal Cord 2011; vol. 49, pp. 679-683. In the acute phase of SCI, there is a risk of respiratory and cardiac failure as disclosed by Song J, Shao J, Qi H H, et al., in an article entitled ‘Risk factors for respiratory failure with tetraplegia after acute traumatic cervical spinal cord injury’, published in Eur Rev Med Pharmaco 2015, vol. 19, pp. 9-14. SCI at the level of the third cervical vertebra or above could cause death or the victim may need a respirator to stay alive. People living with SCI often endure a lifelong disability with complete or incomplete paralysis below the level of injury. Road traffic accidents are the second leading cause of SCI as disclosed by Feng H, Ning G, Feng S, et al., in an article entitled ‘Epidemiological profile of 239 traumatic spinal cord injury cases over a period of 12 years in Tianjin, China’, published in J Spinal Cord Med 2011, vol. 34, pp. 388-394.

Survivors of motor vehicle accidents, usually have severe disability including mental disorders. Most of the damage occurs in the first minutes following the accident due to absence of immediate medical care. It is crucial to limit the time lapse from the accident to initiation of medical intervention, which to this day, even in the most advanced countries, remains unacceptably very long to prevent brain damage. Therefore, researchers are in search for measures to militate against brain damage by providing neuroprotection in the shortest possible time after TBI. It would be preferred that the intervention commences immediately after the accident before medical emergency help arrives. The initial assessment, initiation of stabilization and resuscitation in TBI and cervical spine injury would be needed. Experimental evidence demonstrated that therapeutic temperature modulation with the use of mild induced hypothermia (MIH defined as the maintenance of body temperature at 32° C. to 34° C.) exerts significant neuroprotection and attenuates secondary cerebral insults after TBI. In adult TBI patients, MIH has been used during the acute “early” phase as prophylactic neuroprotection and in the sub-acute “late” phase to control brain edema. When used to control brain edema, MIH is effective in reducing elevated intracranial pressure (ICP) and is a valid therapy of refractory intracranial hypertension in TBI patients.

The present invention generally relates to use of artificial intelligence (AI) to perform initial assessment, initiate stabilization and resuscitation measures by selective modification and control of a patient's body temperature, specifically to the altering of the temperature of the cerebrospinal fluid and brain blood flow. Clinical hypothermia is a condition of abnormally low body temperature generally characterized by a core body temperature of 35 degrees Celsius or less. There are several levels of hypothermia, including mild that is a body core temperature within the range of 32 degrees Celsius to 35 degrees Celsius, moderate between 30 degrees Celsius to 32 degrees Celsius, severe between 24 degrees Celsius to 30 degrees Celsius, and profound—a body temperature of less than 24 degrees Celsius. Most investigators suggest that in severe hypoxic ischemic injury in animal models, the optimum temperature is between 32 degrees Celsius and 34 degrees Celsius as disclosed in an article by Colbourne F, Sutherland G, Corbett D., entitled “Posttraumatic hypothermia: a critical appraisal with implication for clinical treatment’, published in Mol Neurobiol, 1997; vol. 14, pp. 171-201. As body temperature falls below 34 degrees Celsius, there is an increased risk of infection, coagulopathy, thrombocytopenia, renal impairment, and pancreatitis as disclosed by Schubert A, in an article entitled “Side effects of mild hypothermia’, published in J Neurosurg, Anesthes 1995; vol. 7, pp. 139-147; and also by Metz C, Holzschuh M. Bein T. et al., in an article entitled “Moderate hypothermia in patients with severe head injury: cerebral and extracerebral effects’, published in J. Neurosurg., 1996; vol. 85, pp. 533-541. Early initiation of hypothermia is crucial, to be effective, hypothermia needs to be achieved within 2-6 hours of severe hypoxic-ischemic injury possibly begun in the ambulance as disclosed in an article by Bernard S A, Gray T W. Buist M D, et al., entitled “Treatment of comatose survivors of out-of-hospital cardiac arrest with induced hypothermia’, published in N Engl J. Med., 2002; vol. 346, pp. 557-563. The duration of hypothermia for hypoxic ischemic injury depends on the severity of the injury and the delay before the hypothermia is achieved. Within limits, a more severe injury or a longer delay can be compensated for by cooling for longer as disclosed in an article by Gunn A. J. entitled “Cerebral hypothermia for prevention of brain injury following perinatal asphyxia’, published in the Curr Opin Pediatr 2000; vol. 12, pp. 111-115. It is desirable of have a device that will achieve hypothermia in the shortest possible time. Usually in event of an accident with fire outbreak due to combustion there is the converse situation of hyperthermia. Hyperthermia is a clinical condition of abnormally high body temperature, due to exposure to a hot environment or surroundings, overexertion, or fever. Body core temperatures may range from 38 degrees Celsius to 41 degrees Celsius due to conditions such as fever, and may be substantially higher in cases of exposure and overexertion. Hyperthermia is a serious and potentially fatal condition. Hyperthermia may result from systemic inflammatory response, sepsis, stroke, or other TBI. The mechanisms of the effect of hyperthermia on the brain remains unclear, but, there is evidence to indicate that even mild increases in temperature may contribute to neurological deficits. Hyperthermia also increases the cerebral metabolic rate and may deplete cell energy stores. Clinically, hyperthermia could be treated with hypothermia to bring the body temperature to normal.

In clinical practice, induced hypothermia has been used for neuroprotection during cardiovascular surgery, severe cardiac conditions (cardiac arrest, myocardial infarction), neurosurgery, head trauma, subarachnoid hemorrhage, spinal trauma, stroke, thoracic aortic aneurysm repair, and liver transplantation. The mechanisms of action of clinical hypothermia may include blunting of post-insult release of neurotransmitters such as glutamate, reduction of cerebral metabolic rate, moderation of intracellular calcium, prevention of intracellular protein synthesis inhibition, and reduction of free radical formation as well as other enzymatic cascades and even genetic responses. For example, it has been demonstrated that hypothermia produces an attenuation of the release of excitatory neurotransmitters in meningitis and suggest that this treatment may attenuate neuronal stress as disclosed by Irazurta J. E. Olson J. Kiefaber M P, Wong H., in an article entitled “Hypothermia decreases excitatory neurotransmitter release in bacterial meningitis in rabbits’, published in Brain Res 1999; 847:143-148.

Clinical induced hypothermia could be classified as whole body and regional hypothermia, invasive and noninvasive methods. Whole body hypothermia is usually invasive, not only takes a significant amount of time, but also subjects the patient to deleterious effects of hypothermia including cardiac arrhythmias, coagulation problems, increased susceptibility to infections, and problems of discomfort such as profound shivering. Whole body hypothermia is induced by externally cooling the blood and pumping it back to the patient using a bypass machine. This method is an extremely invasive procedure that subjects vast quantities of the patients' blood to pumping for an extended length of time. Such external pumping of blood may be harmful to the blood, and continued pumping of blood into a patient for extensive periods of time, for example, more than one or two hours, is generally avoided. During such procedures anticoagulants for example, heparin may be used, to prevent clotting which may present other undesirable consequences in victims of cerebrovascular accidents.

Prior art has proposed the use of catheter to induce hypothermia. For example, U.S. Pat. No. 5,486,208, to Ginsburg, describes a catheter that is inserted into a blood vessel and a portion of the catheter heated or cooled, transferring heat to the patient's blood and thereby affecting the overall body temperature of the patient. One clear advantage of such devices and methods is that, they may avoid the problems associated with external pumping of blood, however, the method is still invasive and do not eliminate the difficulties that arise when the entire body is subjected to hypothermia. Others have introduced variations of balloons capable of acting as ongoing heat transfer balloons by the continual flow of heat transfer medium through the balloon. U.S. Pat. No. 6,558,412 to Dobak III, describes a flexible catheter that is inserted through the vascular system of a patient to place the distal tip of the catheter in an artery feeding the selected organ. A compressed refrigerant is pumped through the catheter to an expansion element near the distal tip of the catheter, where the refrigerant vaporizes and expands to cool a flexible heat transfer element in the distal tip of the catheter. The heat transfer element cools the blood flowing through the artery, to cool the selected organ, distal to the tip of the catheter.

Regional cerebral hypothermia, by directly cooling the surface of the head. For example, by placing the head in a cooled helmet or shroud, or even injecting a cold solution into the head region. U.S. Pat. No. 6,581,400 to Augustine et al., describes an apparatus for convectively and evaporatively cooling a patient's head. U.S. Pat. No. 6,126,680 to Wass discloses a method and apparatus for convective cooling of the brain in which cooled air is passed over a patient's head resulting in convective cooling of the patient's brain. Other prior art have applied the use of contact pad systems, such as that, disclosed in U.S. Pat. No. 6,197,045, to Carson and U.S. Pat. No. 6,620,187 to Carson et al., for selectively cooling and/or heating bodily tissue. The drawback of using external cooling methods is that it can only lower the temperature of these oxygen-sensitive organs only very slowly at rates of less than 0.05 degrees Celsius/minute (only 3 degrees Celsius/hr). Others have induced hypothermia by use of catheter inserted directly into the cerebrospinal fluid space in the head. For example, U.S. Pat. No. 7,318,834 to Njemanze uses an implanted catheter for cooling and rewarming the CSF.

Prior art, devices were bulky and impractical to use as standalone, except in a medical setting. The helmet designs were not effective, because the insulating qualities of the skull make it difficult to effectively lower brain core temperature, and the blood flow that may fail to provide sufficient heat transfer circulation to the brain itself when the surface of the head is cooled. During direct cooling of the head, patients usually would require general anesthesia, in order to tolerate immersion or direct exposure of the head to a cold solution or cooling surface.

External cooling of the head could be made effective if the cooling is directly above the venous sinuses and CSF spaces. Furthermore, external cooling could be made more effective if the cooling is directly applied over the skin surface in the area neck vessels including arteries and veins that supply the brain and over the cervical spinal cord. Along the cervical spinal cord, the cooling could be effected through slit openings called the anterior median fissure which is a groove along the anterior midline of the spinal cord that incompletely divides it into symmetrical halves also referred to as ventral median fissure. It houses the anterior spinal artery and anterior spinal vein in the anatomy as disclosed by Oichi Y, Hanakita, Takahashi T et al., in an article entitled ‘Morphological patterns of the anterior median fissure in the cervical spinal cord evaluated by computed tomography after myelography, published in Neurospine, 2018, vol. 15 (4), pp. 388-393.

Investigators have reported significant effect of mild hypothermia on uncontrolled intracranial hypertension after severe head injury as disclosed by Shiozaki T, Sugimoto H, Taneda M, et al., in an article titled ‘Effect of mild hypothermia on uncontrollable intracranial hypertension after severe head injury’, published in Journal of Neurosurgery 1993, vol. 79 (3), pp. 363-368. The heat loss effect on the physical fluid characteristics of the CSF would cause a decreased CSF volume and hence reduced intracranial pressure. The fall in intracranial pressure is related to the overall benefit of decreased CSF volume, preserved vasoreactivity and autoregulation as disclosed by Rosner M. in an article entitled ‘Pathophysiology and management of increased intracranial pressure’, in a book by Andrews B T. (ed.) entitled ‘Neurosurgical intensive care’, published by McGraw Hill Inc., New York, pp. 57-112, 1993.

Others have found that mild hypothermia is an effective and simple procedure to improve the cardiac function in patients after cardiac surgery complicated with low cardiac output as disclosed by Zhao Z, Gu T, Zhang Y, Yu L., in an article entitled ‘Effects of mild hypothermia on low cardiac output after cardiac surgery’, published in Zhongguo Wei Zhong Bing Ji Jiu Yi Xue, 2012, vol. 24(4), pp. 219-221.

State of the art research in the automotive industry has focused on active safety and neck injury prevention for intelligent vehicles. The active safety describes systems that use an understanding of the state of the vehicle to avoid or minimize the effects of a crash. The advanced driver assistance systems (ADAS) include braking systems, such as brake assist, traction control systems and electronic stability control systems, which interpret signals from various sensors to help the driver control the vehicle. The state of the art active safety research focuses primarily on sensor-based systems, such as ADAS including adaptive cruise control and collision warning/avoidance/mitigation systems as described by Li F, Liu N, Li H, et al. in an article entitled ‘A review of neck injury and protection in vehicle accidents’, published in Transportation Safety and Environment, 2019, vol. 1, No. 2, pp. 89-105. Until now, there has not been a prior art on automated medical emergency first aid during TBI and neck cervical spine injury in a vehicle accident.

One of the most important safety device systems in vehicles is the deployment of inflated airbags. Airbags comprise stretchable fabrics or other materials that are tightly packed in various locations in the vehicle. Most vehicles have airbags compressed at the front of the dashboard along the side. The crash sensors are very important parts of the airbag system. On impact, the crash sensors trigger the control unit to send signal to the inflator which sets of a chemical charge producing an explosion of nitrogen gas. The airbags fill up with air very quickly to provide a cushioning system for the driver and passengers in the car so that they are not thrown around. The deployment of the airbag happens in an instant, usually within 25 or 50 milliseconds, and it quickly deflates by itself. While this is helpful in cushioning the driver and passengers in a car, it does not prevent injury or death. The most crucial protection to prevent CSI is to prevent hyperextension of the head and neck. However, until now, no airbags are attached to the head rest on seats in vehicles to prevent hyperextension of the head and neck.

A major feature of improving vehicle safety is a timely intervention by the emergency medical services (EMS). Most often in the event of an accident the victims may be too injured to communicate to the EMS that an accident event has occurred. It would be desirable to have a system styled like the concept of the ‘Black Box’ in commercial aircrafts to record audiovideo and photographs of the event from inside the vehicle at the point of crash, so as to raise alarm of the crash with the EMS on duty, providing exact location and the photographs of the victims in the accident for the first responders to be better prepared. However, considering privacy issues only in the event of an accident does the data get recorded and transferred. The storage of the accident events in the ‘Black Box’ of the vehicle would aid later reconstruction of the events that led to the crash.

In the era of AI systems controlling autonomous driving vehicles, it would be essential to have a regulatory framework that requires ‘Black Box’ on vehicles. AI systems are actively being developed by the automotive industry. The AI system monitors dozens of sensors, to identify dangerous situations. The AI system uses the data to draw attention of the driver, or take emergency control of the vehicle in order to avoid an accident. The AI system could implement emergency braking, cross traffic detectors, blind spot monitoring, and driver-assist steering can help avoid accidents, and save lives in the process. The more advanced systems like the Google Waymo's AI software and similar algorithm from Tesla processes data from the vehicles' lidar, radar, high-resolution cameras, GPS, and cloud services to produce control signals that operate the vehicle. The AI software implement powerful AI deep-learning algorithms to accurately predict what objects in the vehicle's travel path are likely to do, using images from eight cameras, an array of ultrasonic sensors, sonar, forward-facing radar, and GPS, to turn sensory data into vehicle control data. Although these AI systems could serve well as co-pilot to the human driver, it is more desirable for the AI system to be involved in the resuscitation of the driver and passengers in the event of an accident.

### SUMMARY OF THE INVENTION

The present invention provides a medical emergency first aid during accidents using cervical spine collar to provide stabilization, resuscitation and initiation of mild to moderate hypothermia in victims of traumatic brain (TBI) and cervical spine injury (CSI). In one embodiment it could be used with protective helmet with cooling apparatus to induce mild to moderate hypothermia of the brain to prevent deleterious effects of TBI. The neck collar device of the present invention could be inserted manually by an assistant as well as automatically by an artificial intelligence (AI) system. In one embodiment, the present invention provides a medical emergency first aid safety system in vehicles during accidents using AI system to automatically provide initial assessment, initiate stabilization and resuscitation of victims of TBI and CSI. The AI system on detection of a crash automatically uses the pre-determined anthropometric variables and position-sensing of the head and neck including measurements of neck circumference to insert a splint using an automatic cervical spine collar deployed from the head rest of the vehicle seat.

It is an object of the present invention for the AI system to use audiovideo guidance to instruct and assist victims to properly place the device for initial stabilization and resuscitation in the event of TBI and CSI in the aftermath of the accident. The AI system uses the pre-departure demonstration to acquaint the driver and passenger on the safety procedure.

It is an object of the present invention to use deep machine learning for the AI system to implement image pattern recognition algorithm for determination of the position and size of the head, neck circumference and other parameters in order to splint the persons more appropriately to fit.

One particular object of the present invention is the use of AI system for automation of initial assessment that provide information about the neck injury mechanisms according to load directions, simulations methods, and threshold by means of impact intensity, load intensity and stress/strain conditions.

Another object of the present invention is to detect the crash, automatically determine head, neck and body position, neck circumference suitable to insert a splint using an automatic cervical spine collar attached to the head rest of the seat.

Another object of the present invention is to simultaneously inflate a system of inner bladders filled with air coolant for cooling the neck and back of the head region to induce mild to moderate hypothermia.

A further object of the present invention is on detection of a crash to automatically inflate a mini-airbag from the head rest to prevent hyperextension of the head and cervical spine. The ensuing hyperflexion could be prevented by the automatic deployment of the front airbag on the steering.

An object of the present invention is to determine the state of the driver and passengers in the immediate aftermath of the vehicle accident and to communicate by audiovideo and photographs including GPS location to the emergency medical service (EMS) team, police, fire fighters for immediate evacuation through wireless mobile phone service or other wide area network (WAN) system. To store the audiovideo information in the vehicle ‘Black Box’ for use in future investigation of the accident. Orthopedic surgeons reviewing the video tapes of the victims in the vehicle during the accident could better appreciate the nature of the injuries sustained by each person for timely and proper intervention.

The present invention has the added advantage of selectively cooling the brain to prevent severe pain, death and the adverse neurologic sequel from TBI and CSI. Even when death or severe injury does not occur, several victims report significant neuropsychologic deficits resulting from TBI after vehicle accidents, which could be significantly reduced by application of the present invention.

The present invention may relate to the effects of mild hypothermia to significantly reduce the intracranial pressure and increase the cerebral perfusion pressure. As it has been demonstrated that, mild hypothermia is a safe and effective method to control traumatic intracranial hypertension and to improve mortality and morbidity rate. The present invention would permit the use of hypothermia to desirably alter the major indices of cerebral autoregulation namely cerebral blood flow and mean arterial blood pressure.

In the aftermath of a vehicle accident, cardiovascular shock could ensue leading to reduction in cardiac function. The present invention implements hypothermia to raise low cardiac output and blood pressure that will help overcome shock.

It is the object of the present invention to provide an automated cervical collar to therapeutically help realign the spinal cord and relieve the severe effects of pain, strains, sprains and whiplash in the aftermath of a vehicle accident.

It is an object of the present invention to prevent brain damage by incorporating apparatus (AIAutoCoolHelmets) for application of mild induced hypothermia (MIH defined as the maintenance of body temperature at 32° C. to 34° C.) into the protective helmets such as those used in car-racing, military vehicles and aircrafts. The same type of helmets could be adapted as standalone for use in sports, motorbikes, construction, pilots, and any other areas where protective helmets are worn for improved safety because of TBI.

The special embodiment of this invention is illustrated in the specifications, it includes block and schematic diagram for the format of the instrumentation, and how the system functions is shown by way of example. The human involved will be referred to as “driver or passenger” by way of example. Although in some cases the invention could be adapted for use in driverless vehicles. To illustrate the concept, we would apply the use of the device in a vehicle with AI system, just as an example, which should not limit the use of this device since it can be used for several other applications including as a standalone device with same features that is manually inserted on the victim of accident. The foregoing summary of the present invention with the preferred embodiments should not be construed to limit the scope of the invention. It should be understood and obvious to one skilled in the art that the embodiments of the invention thus described may be further modified without departing from the spirit and scope of the invention.

These and other objects of the invention may become more apparent to those skilled in the art upon reviewing the description of the invention as set forth hereinafter, in view of its drawings.

### BRIEF DESCRIPTION OF THE SEVERAL VIEWS OF THE DRAWINGS

FIG. 1 shows the mechanism of heat transfer to the CSF (FIG. 1A) and vessels (FIG. 1B) in the head and neck region.

FIG. 2, shows the head and neck region where the AIAutoCoolCollar is applied.

FIG. 3 A-F, shows the assembly of the device (AIAutoCoolCollar) and the various parts.

FIG. 3A, shows one assembly of the device as a standalone neck collar for initial stabilization and external hypothermia and could be adapted for use in a vehicle.

FIG. 3B, shows the schematic diagram of the inside of the device with central cooling bladder (black) inflated by coolant for external hypothermia and side bladders (gray) inflated with normal air.

FIG. 3C, shows the inside surface of the device with the central cooling bladder (black) and side air bladders (gray) placed on the C-arm of the device to move in a semi-circle to close the collar for neck stabilization.

FIG. 3D, shows the front view of one embodiment of the splint with the device (AutoCoolCollar) put in place as first aid, manually, by a conscious driver or an assistant.

FIG. 3E, shows the front view of another embodiment of the splint with the device (AIAutoCoolCollar) in place as first aid by the AI system.

FIG. 3F, shows the front exploded view of feature 47, showing the ultrasonic sensor and display of arterial pulsation.

FIG. 4, shows a schematic diagram of the side view of the device inside a head rest. The head rest airbag is shown in exploded view.

FIG. 5 A-H shows the operational sequence of the device (AIAutoCoolCollar) during a motor vehicle accident.

FIG. 5A, shows the time preceding a crash, the driver's head and neck are in normal position.

FIG. 5B, shows that at the moment of impact the head and neck are in flexion.

FIG. 5C, shows the next stage of head and neck hyperextension.

FIG. 5D, shows the final stage of head and neck hyperflexion.

FIG. 5E, shows the head rest fitted with the device and the head and neck of the driver in normal position.

FIG. 5F, shows the moment of crash when the device starts to be deployed.

FIG. 5G, shows the driver with the head and neck in hyperextension as the device deploys from the head rest. The front airbag deploys and forces the head and neck backwards preventing hyperflexion, while the head rest back airbag prevents hyperextension.

FIG. 5H, shows the driver with the splint accomplished and the device detached from the head rest.

FIG. 6, shows the flow chart of the AI program that operates the device and other safety features.

FIG. 7, shows the driver with the device deployed in the vehicle moments after the impact.

FIG. 8 A-D shows areas of distribution of blood flow for heat transfer from the detailed parts of the assembly of the device (AIAutoCoolHelmet).

FIG. 8A, shows the venous sinuses for blood flow distribution in the area covered by the brain cooling system.

FIG. 8B, shows a protective helmet (AIAutoCoolHelmet) that could be used by race car drivers as a component of the present invention, and is applicable as a standalone for motorbikes.

FIG. 8C, shows another embodiment of a protective helmet adapted as standalone device according to the teachings of the present invention for use in sports such as in American football.

FIG. 8D, shows the interior cooling apparatus of the protective helmet.

FIG. 9, shows a race car driver with the AIAutoCoolCollar and AIAutoCoolHelmet fully deployed.

### DESCRIPTION OF THE PREFERRED EMBODIMENT

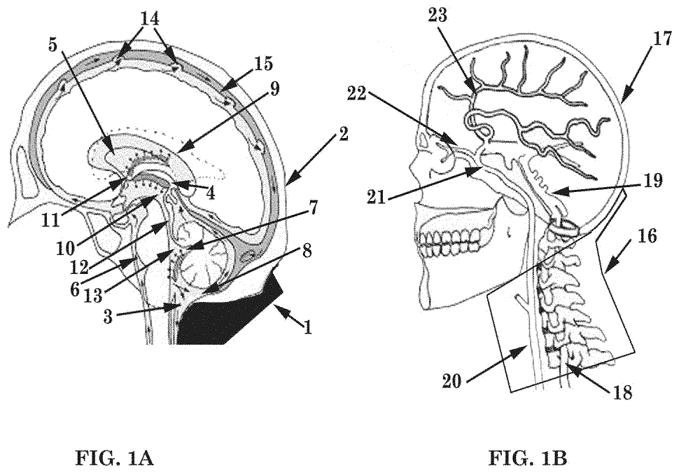

FIG. 1 shows the mechanism of heat transfer to the CSF (FIG. 1A) and vessels (FIG. 1B) in the head and neck region. FIG. 1A, shows the mechanism of transfer of heat to the CSF in the head and neck region. The aim of the system of the device 1 is to provide direct heat exchange through the skin of the back of the head and neck 2 with the CSF compartment 3 cooling the CSF that baths the brain tissue which in turn will slow cerebral metabolic rate to conserve energy in the neuronal cells, and would have a positive influence on temperature regulation, decreasing CSF volume leading to reduction in intracranial pressure (ICP), increasing cerebral perfusion pressure (CPP) and preserving cerebral autoregulation mechanisms. The heat exchange is accomplished due to the pulsatile flow of CSF. The choroid plexus 4 of each of the lateral ventricles 5 produce the CSF. The CSF flows through the ventricles and into the subarachnoid space 6 through the median 7 and lateral 8 apertures. The heat exchange follows the pulsatile activity of arteries within both thalami as disclosed in an article by Njemanze P C, Beck O J., entitled ‘MR-Gated Intracranial CSF dynamics: evaluation of CSF pulsatile Flow’, published in AJNR 1989; vol. 10, pp. 77-80. The pulsatile activity described as the thalamic pump, pumps CSF flowing in from the lateral ventricles 5 on both sides of the mid corpus callosum 9, into the third ventricle 10, through the interventricular foremen of Monroe 11. The CSF flows into the aqueduct of Sylvius 12 into the Fourth ventricle 13. The CSF is absorbed into the dural venous sinuses via the arachnoid villi 14 located beneath the arachnoid mater 15. The heat transfer through CSF flow provides cooling to the entire structures of the brain.

FIG. 1B, shows the mechanism of transfer of heat to the vessels in the head and neck region. The device also facilitates heat exchange through the arteries of the brain. The device is applied directly on the skin overlying the neck 16 and back of the head regions 17, in direct contact with both vertebral arteries 18, that supply the basilar artery 19, into the posterior circulation of the brain. The vertebral arteries 18 also form at the region of the medulla oblongata, the anterior spinal artery which supplies the anterior portion of the spinal cord to facilitate heat exchange with the spinal cord. Blood flow from both common carotid arteries 20, supply to the internal carotid arteries 21, which in turn supply the anterior cerebral arteries 22 and middle cerebral arteries 23, that provides blood circulation in the circle of Willis to the entire brain.

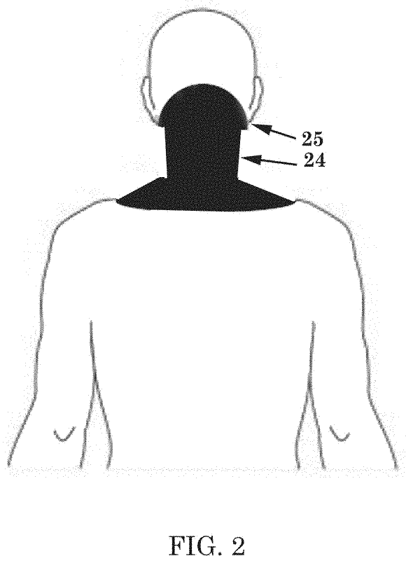

FIG. 2, shows the head and neck region where the AIAutoCoolCollar is applied. The device is placed over the neck 24 and occiput 25 in the back of the head.

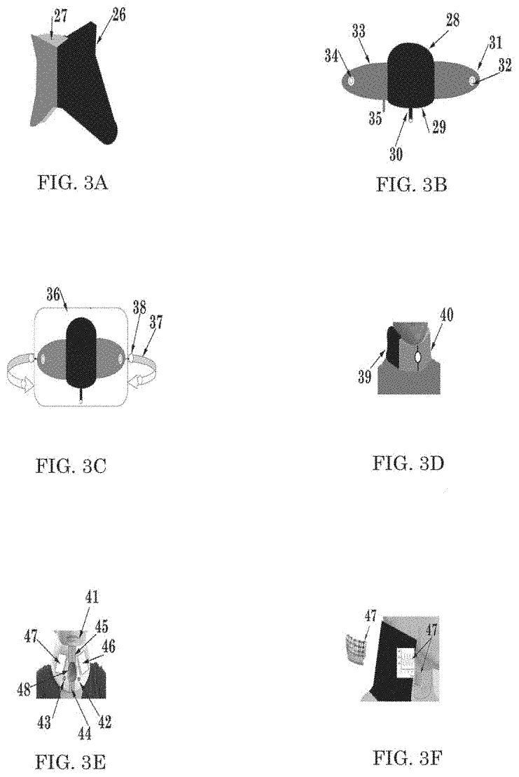

FIG. 3 A-F, shows the assembly of the device (AIAutoCoolCollar) and the various parts. FIG. 3A, shows one assembly of the device as a standalone neck collar for initial stabilization and external hypothermia and could be adapted for use in a vehicle. There is the back side (black) 26 that is cooled overlying the back side of the neck and head (covering vessels and CSF compartment). The front side of the head and neck is not cooled (gray) 27. Materials for assembling the neck collar for this device are readily available and could be obtained from a number of companies for example Timago International Group, Poland, the manufacturer of the Philadelphia Cervical Collar. FIG. 3B, shows the schematic diagram of the inside of the device with central cooling bladder (black) inflated by coolant for external hypothermia and side bladders (gray) inflated with normal air. The inflation could be both by manual squeezable bulb or automatically controlled pump to the point that it conveniently fits to achieve maximum stabilization of the neck. The central cooling bladder (black) has the head end curvature 28, and the shoulder support end 29, for external hypothermia. The cool air is pumped through a nozzle 30, to inflate the central cooling bladder. In the manual mode the Velcro surface allows manual adjustment to the size of the neck of the victim. The automatic mode controlled by the AI system utilizes the video images and measurements taken for initial assessment of the accident victims and their positions in the vehicle, for the splinting process. The anthropometric variables are used to make adjustments that will allow automatic fitting of the device neck collar to fit the person appropriately. The AI system obtains visual data from inside of the vehicle using the camera mounted on the rear view mirror or elsewhere in the front dashboard. The information includes among others the head position and neck circumference of the driver and passengers in the vehicle. The head rest of the seat fitted with the device has adjustable height to match persons of different heights while seated. The inflation of the bladder is gauged to match the size of the neck for neck collar stabilization. The central cooling bladder 29 is inflated through a nozzle 30, for supply of coolant to a preprogrammed gauge according to neck circumference. This is accomplished by the AI system by regulating how long and at what rate the valve lets in the coolant or air into the bladder. The supply could be from the coolant of the air-conditioning system of the vehicle or a standalone coolant canister. The left side air bladder 31 with locking button 32 and the right side air bladder 33 with locking button 34 are filled through an air nozzle 35, from a supply within the vehicle or standalone canister through a valve regulator.

FIG. 3C, shows the inside surface of the device with the central cooling bladder (black) and side air bladders (gray) placed on the C-arm of the device to move in a semi-circle from both sides to close the collar for neck stabilization. The device is contained in a head rest 36, and self-deploys under control of the AI system through openings on both sides with automated levers that perform a semicircular movement of the left C-arm 37 with lock 38, to meet the opposite right side.

FIG. 3D, shows the front view of one embodiment of the splint with the device (AutoCoolCollar) put in place as first aid, manually by a conscious driver or an assistant. A situation could arise when the driver is fully conscious or that the AI system malfunctions or was not even programmed. The device cooling bladder 39 and air bladder 40, could be manually put in place. If in the vehicle, the driver or passenger-assistant could manually detach the device from the head rest and attach it to the victim. The Velcro surface allow adjustment to neck size.

FIG. 3E, shows the front view of another embodiment of the splint with the device (AIAutoCoolCollar) in place as first aid placed by the AI system. The device upper end is beneath the lower jaw 41. The movement of the C-arms are guided by the camera and optical tracking units of the AI system on the left side 42 and wireless communication such as Bluetooth on the side right 43, such that, the lower 44 and upper 45 locking units slide together appropriately. Beneath the lower 44 and upper 45 locking units are spaces provided for mini battery storage to independently power the electronics in the cervical collar. The battery in one embodiment is made rechargeable. The reflective surfaces on the left 46, reflects light and makes it easy to track victims of accidents especially at night. In one embodiment of the present invention on the right side 47 is an in-built mini wearable ultrasonic sensor for arterial pulse pressure with screen display and a loud speaker to relate the vital signs of the victim for proper triage when there are a number of victims. In another embodiment, the device has in place of the reflective surface on the left 46, a display of oxygen saturation measured using a standard pulse oximetry device placed above the jugular vein, which is a non-invasive method to measure arterial oxygen saturation level. A pulse oximetry device includes a sensor that measures light absorption of hemoglobin and represents arterial SpO2. The basis is that, oxyhemoglobin and unoxygenated hemoglobin absorb light differently. The pulse oxymeter sensor 46 measures the relative amount of light absorbed by oxyhemoglobin and unoxygenated (reduced) hemoglobin and compares the amount of light emitted to light absorbed. This comparison is then converted to a ratio and is expressed as a percentage of SpO2. Pulse oximetry is described in detail by Jubran A. in an article entitled, ‘Pulse Oximetry’, published in Crit Care, 2015; vol. 19(1), p. 272 (doi:10.1186/s13054-015-0984-8). There is a large ventilation central hole 48, and a couple of small ventilation holes on the back surface.

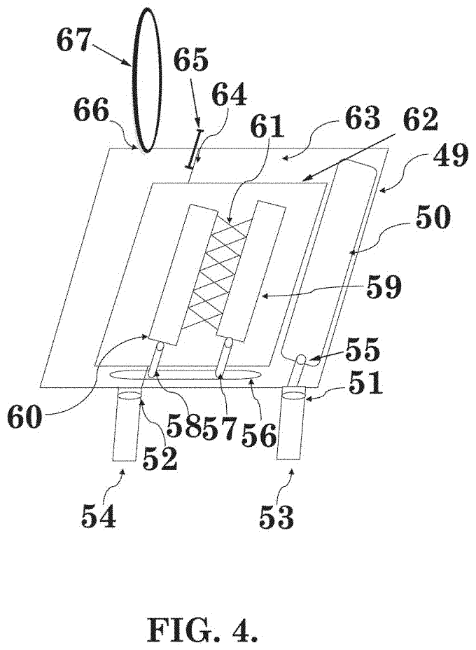

FIG. 3F, shows the front exploded view of feature 47, showing the ultrasonic sensor and display of arterial pulsation. This wearable ultrasonic device that is placed on the skin surface 47 over the carotid artery has been described in detail by Wang C. et al., in an article entitled ‘Monitoring of the central blood pressure waveform via a conformal ultrasonic device’ published in Nature Biomedical Engineering 2018, vol. 2, pp. 687-695. Both the pulse sensor and the microphone are activated by the crash sensor, and could be shut off manually. The vital signs could include blood pressure and pulse measurements. In some other embodiment infra-red oxygen saturation measurement is displayed. These vital signs help to characterize the cardiovascular status of the victim and prioritize victims in serious conditions during the triage by the EMS. The AI system has the capacity to telemeter the vital signs from victims through wireless communication including use of Bluetooth technology. FIG. 4, shows a schematic diagram of the side view of the device inside a head rest. The head rest airbag is shown in exploded view. The front end of the head rest 49 encloses the central cooling 50 bladder and side air bladders which come in contact with the skin of the person. The device is suspended on left 51 and right 52 detachable rings which are held in place by the left 53 and right 54 suspension rods which could automatically disengage the device from the head rest as soon it is fully deployed. The coolant is pumped through a nozzle 55 to fill the central cooling bladder in case of a crash. The C-arms of the collar are put in place by a system of robotic type levers which comes out from a groove 56 within the head rest, which move the left 57 and right 58 levers in a semi-circular movement to bring the left 59 and right 60 C-arms together at the front systems could be obtained from a number of companies such as Sparton Inc. in Schaumburg, Ill., USA. The separation of the left 59 and right 60 C-arms is facilitated by a spring system 61 situated between them within the inner casing 62 which is placed within the outer casing 63 of the head rest. The front end of the head rest comprising the device separates automatically from the back end at the borderline 64, and could be also done manually by pressing the unlocking button 65. The back of the head rest has a thin slit opening 66 through which the head rest airbag 67 could on impact deploy from underneath, to prevent excessive hyperextension of the head of the passenger or driver.

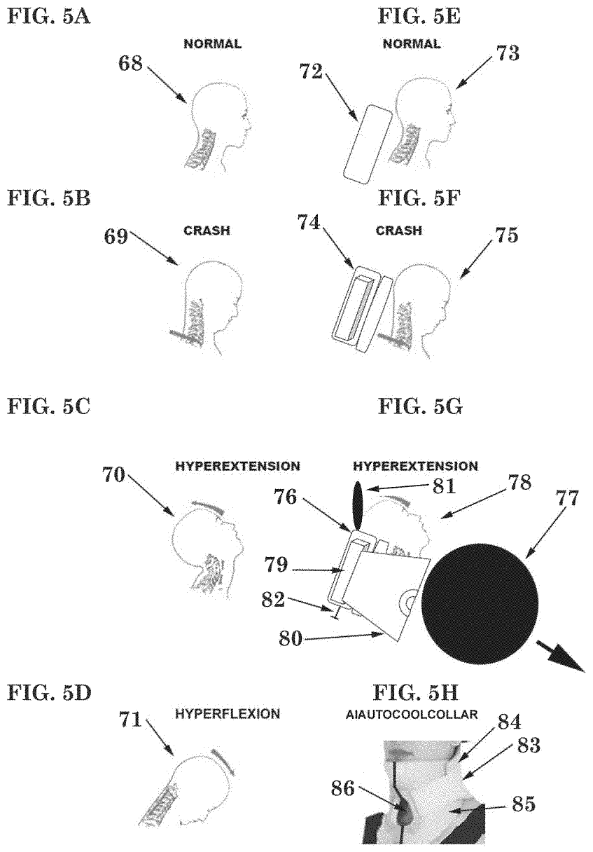

FIG. 5 A-H shows the operational sequence of the device (AIAutoCoolCollar) during a motor vehicle accident.

FIG. 5A, shows the time preceding a crash, the driver's head and neck are normal position 68. FIG. 5B, shows that at the moment of impact the head and neck are in flexion 69.

FIG. 5C, shows the next stage of head and neck hyperextension 70.

FIG. 5D, shows the final stage of head and neck hyperflexion 71.

FIG. 5E, shows the head rest 72 fitted with the device and the head and neck of the driver 73 in normal position. The AI system is designed to work in a vehicle with driver or that is driverless. The AI system performs a pre-departure safety briefing, demonstration and exchange with the persons in the vehicle in a similar way as pre-flight safety demonstration in commercial airplanes. The pre-departure information includes photos, identity, age of the persons, telephone numbers etc. The information of the demonstration includes the safety procedures related to the deployment of the AIAutoCoolCollar and AIAutoCoolHelmet. Both components of the present device could be used independently or together as in race cars. The persons are instructed using a video guide on the deployment procedure and how to keep the head and neck in position. Assurances are given that the AI system has measured their neck circumference and the splint would be fitted to the appropriate neck size and there is no possibility of strangulation. Furthermore, it would be emphasized that, the AI system would only deploy if the crash intensity is high and the video from the inside of the vehicle indicates that there is a high risk of a head and neck injury. The AI system could be built using commercially available Artificial Intelligence platforms and could also use Deep Learning software and Machine Learning software. Deep learning software tools are commercially available and some are open source software written in languages like Python, Cython, C+, C++, CUDA, Java and others, that can run on the usual common platforms like Linux, Mac OS and Windows. Some aspects that involve facial recognition software could also use commercially available software like DeepFace from Facebook, Amazon Rekognition, Local Binary Pattern Histogram (LBPH) and others. FIG. 5F, shows the moment of crash when the device starts to be deployed 74. The head and neck of the driver is moved forward on rear impact 75.

FIG. 5G, shows the driver with the head and neck in hyperextension as the device deploys from the head rest 76. The front airbag 77 deploys and prevents the ensuing hyperflexion, but quickly collapses forward as shown by thick black arrow. The head and neck are forced backwards in hyperextension 78. As the head and neck remain in hyperextension 78, the device deploys sideways 79, and the left and right C-arms 80 are rolled into place to close at midline. The immediate expansion of the back airbag 81 from the head rest prevents hyperextension. Once deployed, the device separates automatically from the head rest, or a manual button 82 could also be needed for manual separation in case the electronic separation was not accomplished.

FIG. 5H, shows the driver with the splint accomplished and the device detached from the head rest. The device cools the back and sides of the neck 83, up to the area of the occiput 84. The frontal area of the neck 85 is at normal body temperature. There is a central vent opening 86 for aeration.

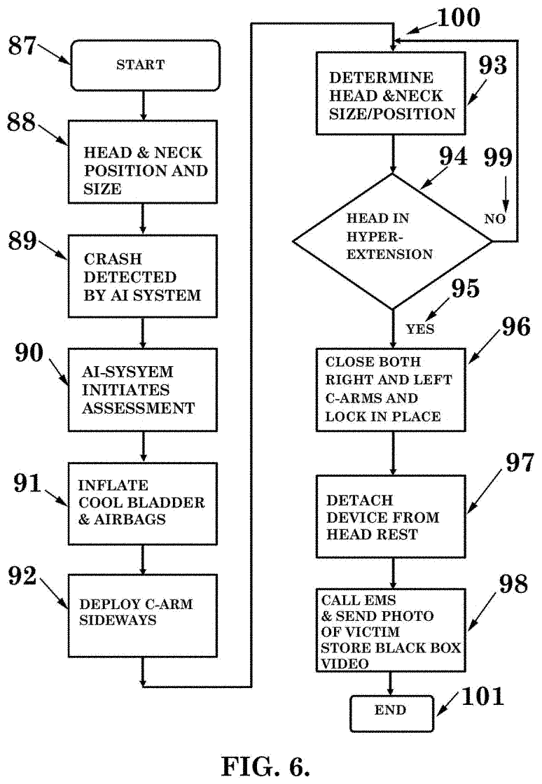

FIG. 6, shows the flow chart of the AI program that operates the device and other safety features. The AI system starts 87 with a pre-departure demonstration to show the safety feature in the vehicle and explain how the device works. The AI system determines the position of the head and neck circumference 88, and alongside other data proceeds to adjust the head rest so the device can be safely deployed in the event of a crash. AI system determines when a crash occurs from a host of crash sensors 89. The AI system performs an initial assessment 90 of the audio-video images from inside the vehicle, to determine if there was a risk of TBI and/or CSI. The AI system initiates deployment of the device by inflating the head rest airbag 91. In situations where the driver has the helmet device as with race car driver involved in a crash, the AI system wirelessly activates the crash sensor on the helmet to begin cooling of the head. The C-arm of the device begins to deploy 92 side ways in preparation to begins full cooling of the head and neck region. The AI system determines the current position and size of the head and neck 93. If the head and neck are in hyperextension 94, the AI system proceeds to splinting 95 by inserting the C-arms in place 96. Thereafter the device is automatically detached from the head rest 97. The AI system calls the EMS 98 and other relevant first responders. It sends images of the accident and vital signs to convey the state-of-being of the victims so as to prepare the first responders for adequate first aid. The event is recorded and stored on audiovideo tapes in the vehicle's ‘Black-Box’. However, if the head of the person was not in hyperextension 99 the AI system repeats from the prior step 100 to determine the position and size of the head and neck 93, if in hyperextension 95 it then proceeds with the successive steps until the end 101. In another embodiment, an assistant could manually take the cervical collar from within the head rest and place it on the victim, especially in the absence of an automatic AI system.

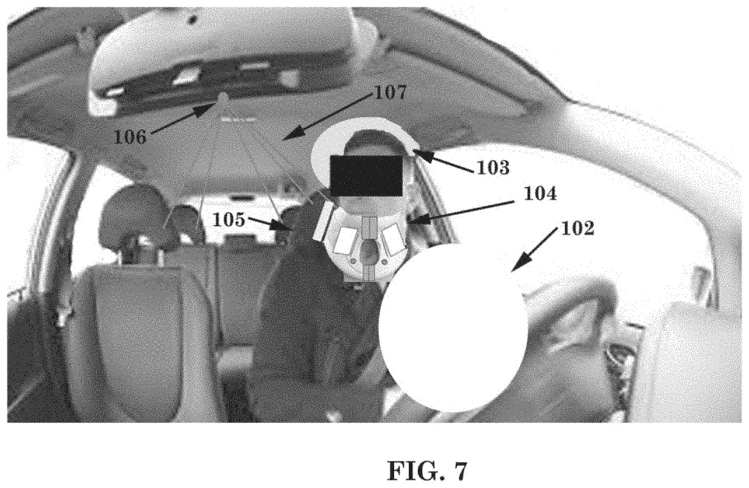

FIG. 7, shows the driver with the device deployed in the vehicle moments after the impact. The front air bag 102 deploys and prevents the driver from going into hyperflexion, while the back head rest airbag 103 prevents hyperextension. The driver is in position with head on the head rest and neck slightly extended for deployment of the device 104 from the head rest 105. The deployment of the device is guided by the AI system using visual input data from the camera 106, and wireless communication such as infra-red 107.

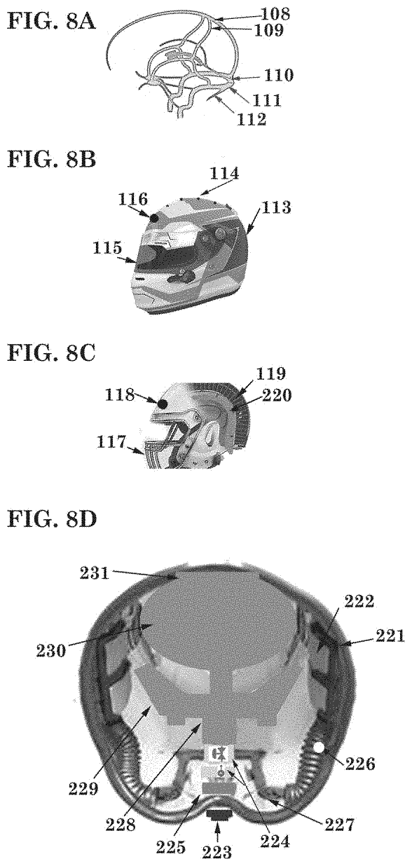

FIG. 8 A-D shows areas of distribution of blood flow for heat transfer from the detailed parts of the assembly of the device (AIAutoCoolHelmet).

FIG. 8A, shows the venous sinuses for blood flow distribution in the area covered by the brain cooling system. The veins of the brain in this region include the superior sagittal sinus 108, the superior anastomatic vein of Trolard 109, the confluence of sinuses 110, the transverse sinus 111, and the occipital sinus 112. The cooling is transferred through cerebral blood flow in these superficially lying veins throughout the entire brain and to the other organs.

FIG. 8B, shows a protective helmet (AIAutoCoolHelmet) that could be used by race car drivers as a component of the present invention, and is applicable as a standalone. The outer shell of the helmet 113 serves to protect the head with some ventilation holes 114 for aeration. The front face shield 115 protects the face and eyes. There is an outer “crash” sensor 116, which are both mechanical and electronic sensors that sense significant vibration and acceleration to activate the interior cooling system. The “crash” sensor 116 could be activated automatically by the AI system signaling a crash or manually by an assistant.

FIG. 8C, shows another embodiment of a protective helmet adapted as standalone device according to the teachings of the present invention for use in sports such as in American football. There is a unique face mask 117. A “crash” sensor 118 is positioned on the forehead to detect significant change in vibration and acceleration. The inner core layer of the helmet 119 which lies below the shell provides support for the inner padding with the inner cooling system bladders 220 lying above it.

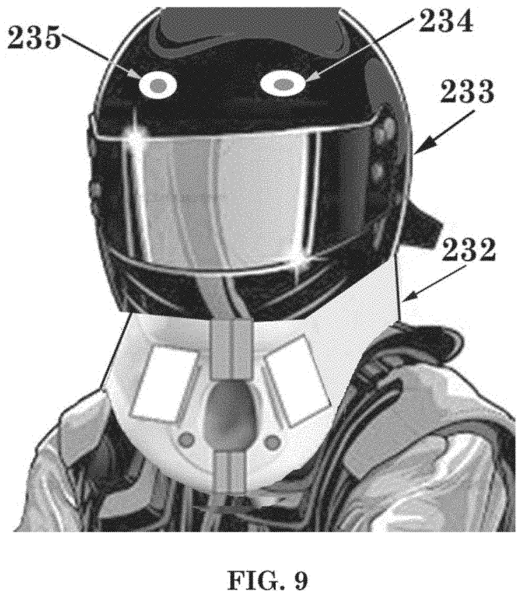

FIG. 8D, shows the interior cooling apparatus of the protective helmet. The coolant is stored in a reservoir 221 under a thermal padding insulator 222, connected to a system of cooling bladders for heat transfer. Once a crash occurs, the crash sensor 223 triggers the automatic gas/liquid valve 224 to open and permit heat transfer to a measure. Both the crash sensor and valve function mechanically and electrically, powered by a small battery 225. In one embodiment, in the event of a crash, the crash sensor activates the ultrasound pulse sensor 226, placed above the temporal artery and connected to a small screen to display arterial pulsation as shown in FIG. 9, and a loud speaker, to show the vital signs of the victim. The pulse sensor 226 is battery powered and is only activated by the crash sensor 116, and could be shut off manually. The coolant from the reservoir 221 passes through conduits and pipes 227 into the system of inner cooling bladders along the midline 228 overlying the superior sagittal sinus, the transverse 229 over the superior anastomotic vein of Trolard, the occiput 230, and at the back of the neck region 231. The coolant could be liquid or gas, and should be a natural coolant that is environmentally friendly and has no ozone depletion potential (ODP), and very low or zero global warming potential (GWP). The most common natural coolants are carbon dioxide, propane, isobutene, profane and ammonia. The choice of coolant is influenced by critical temperature and critical pressure parameters selected according to specific use and preferences including cost.

FIG. 9, shows a race car driver with the AIAutoCoolCollar and AIAutoCoolHelmet fully deployed. The device AIAutoCoolCollar 232 is used to splint the neck in case of cervical spinal injury. The AIAutoCoolHelmet 233 protects against traumatic brain injury. The AI system uses the crash sensors 234 to triggering cooling and vital signs monitoring, which the optical sensor 235 provides head position sensing.

What is claimed is:

1. A safety system method, wherein a cervical spine collar comprising the inside cooling apparatus for mild to moderate external hypothermia of the cerebrospinal fluid and cerebral blood flow of the spine and brain, used to initiate a splint for immediate neck stabilization and resuscitation after cervical spinal injury and traumatic brain injury.

2. The method of claim 1, wherein the cervical spine collar could be manually or automatically deployed.

3. The method of claim 1, wherein the cervical spine collar is integrated with vital signs monitoring device, screen display, microphone and loud speaker powered by a battery to provide the state of health of the accident victim.

4. The method of claim 1, wherein the cervical spine collar is placed into the head rest of the seat of the vehicle, and in the absence of the automatic system for cooling and deployment could be manually inserted on an accident victim by an assistant.

5. The method of claim 1, wherein the crash sensor of the vehicle initiates the deployment of the cervical spine collar.

6. The method of claim 1, wherein the cervical collar is deployed automatically by the artificial intelligence system of the vehicle after obtaining information from the crash sensors.

7. The method of claim 1, wherein the cervical collar is deployed automatically by the artificial intelligence system of the vehicle using information obtained from pre-departure safety briefings of the persons in the vehicle.

8. The method of claim 1, wherein the artificial intelligence system, monitors the vital signs of the victim with sensors on the cervical collar for assessment of the health status.

9. The method of claim 1, wherein the artificial intelligence system of the vehicle, stores photo images, location information, audiovisual and vital signs recordings of the accident victims in a ‘black box’ storage device and could selectively telemeter the information to emergency medical service.

10. The method of claim 1, wherein the artificial intelligence system after automatic deployment of the cervical collar initiates a complete separation of the cervical spine collar from the head rest of the vehicle seat.

11. A safety system method, wherein a protective helmet with crash sensor and inside cooling apparatus for mild to moderate external hypothermia of the brain to prevent the deleterious effects of traumatic brain injury.

12. The method of claim 11, wherein the protective helmet is adapted to be used to prevent traumatic brain injury in a vehicle accident.

13. The method of claim 11, wherein the protective helmet is adapted to be used to prevent the deleterious effects of traumatic brain injury in sports accident.

14. The method of claim 11, wherein the protective helmet is adapted to be used to prevent the deleterious effects of traumatic brain injury for military applications.

15. The method of claim 11, wherein the protective helmet is adapted to be used by construction workers.

16. The method of claim 11, wherein the protective helmet is adapted to be used by car racing drivers.

17. The method of claim 11, wherein the protective helmet has vital signs monitoring device to report the health status of the victim in the event of traumatic brain injury.

18. The method of claim 11, wherein the protective helmet when used in a vehicle are controlled by the artificial intelligence system which collects data from the crash sensors to initiate mild to moderate hypothermia of the brain in the event of traumatic brain injury during a vehicle accident.

19. A safety system method, wherein the crash sensor initiates expansion of the airbag located at the back of the head within the head rest of the seat of the vehicle to prevent hyperextension of the head and cervical spine of the victim.

20. The method of claim 19, wherein the artificial intelligence of the vehicle deploys the head rest airbag based on information on the severity of the crash from the crash sensors.
